(19) United States
(12) Patent Application Publication
(10) Pub. No.: US 2026/0146990 A1
(43) Pub. Date: May 28, 2026

(54) SYSTEM AND METHOD FOR PREDICTIVE MODELING OF CELLULAR SYSTEMS

(71) Applicant: George Mason University, Fairfax, VA (US)

(72) Inventors: Nadine Kabbani, Germantown, MD (US); Patricia Sinclair, Annandale, VA (US); William Jeffries, Centreville, VA (US)

(73) Assignee: George Mason University, Fairfax, VA (US)

(21) Appl. No.: 19/400,638

(22) Filed: Nov. 25, 2025

Related U.S. Application Data
(60) Provisional application No. 63/724,790, filed on Nov. 25, 2024.

(51) Int. Cl. G01N 33/50; G01N 21/64; G06T 7/00; G06V 10/774; G06V 20/69

(52) U.S. Cl. CPC G01N 33/502; G01N 21/6428; G01N 21/6456; G01N 33/5026; G01N 33/5079; G06T 7/0012; G06V 10/774; G06V 20/695; G01N 2021/6439; G06T 2207/20044; G06T 2207/20081; G06T 2207/30024

(57) ABSTRACT

A system and method for predictive modeling of cellular systems assesses compound effectiveness on cellular metabolic and inflammatory states. Cellular samples are exposed to metabolic MRX sensors detecting oxidative stress, reporting cytoskeletal organization, or monitoring mitochondrial morphology, dynamics, or compartment-specific redox activity. Images of MRX sensor-labeled samples are captured using an imaging device and analyzed by trained predictive models to determine compound impact on immunological activation, inflammatory status, oxidative stress, mitochondrial function, and bio-energetic state. The system supports multisensor integration and spectral unmixing to generate multidimensional metabolic or ROS related MRX fingerprints that characterize cellular phenotypes, drug responses, and enable comparison to reference states. Preprocessing modules perform image normalization, augmentation, segmentation, and feature extraction to enhance model performance. Predictive models classify treatment conditions, identify perturbation-specific signatures, and enable compound ranking or mechanism-of-action inference. The system provides an objective, scalable, and high-content platform for drug screening, toxicity assessment, and inflammation research.

### CROSS-REFERENCE TO RELATED APPLICATIONS

This application claims benefit to Provisional Application No. 63/724,790 filed Nov. 25, 2024, the contents of which are herein incorporated by reference.

### BACKGROUND OF THE INVENTION

### Field of Endeavor

The present invention relates to biomedical imaging and predictive modeling and, more particularly, to improved systems and methods for detecting and quantifying cellular inflammation and oxidative stress in the context of drug discovery.

### Background of Related Art

Inflammation is a fundamental biological process that protects the body against pathogens, toxins, and tissue injury. However, dysregulated or chronic inflammation contributes to the onset and progression of numerous diseases, including neurodegenerative disorders, autoimmune conditions, metabolic disease, and cancer. A key feature of inflammatory activity is the disruption of cellular redox balance and mitochondrial homeostasis. Mitochondria act not only as metabolic hubs and central regulators of oxidative stress important for immune signaling. During inflammatory responses, mitochondrial dysfunction is manifested as altered membrane potential, fragmentation of the mitochondrial network, impaired oxidative processes, and elevated production of reactive oxygen species (ROS). The ability to accurately detect and quantify these intracellular inflammatory states is essential for evaluating the efficacy, mechanism of action, and safety of new therapeutic compounds. Cell-based assays that track inflammatory markers including ROS generation and cytokine expression are central to drug development and the characterization of immune modulating compounds. In addition, high-content imaging technologies when strategically combined with metabolic and redox-sensitive sensors allow for direct insight into how candidate compounds may modulate cell disease through inflammation. The integration of rationale-based approaches can support the development of more predictive, physiologically relevant models and tools for drug discovery.

Cannabinoids such as plant-derived phytocannabinoids are an important class of compounds found in the Cannabis plant (Cannabis sativa) that interact with the body's cannabinoid receptor system. Cannabidiol (CBD) is a prototype phytocannabinoid with strong anti-inflammatory and anti-oxidative stress properties that have been well documented in cells, animal models, and humans. In experimental studies, ROS dynamics can be measured directly using validated sensors such CellROX and MitoROS that are fluorescent dynamic ROS indicators in cytoplasm and nucleus as well as mitochondria, respectively. The effect of CBD on ROS levels, spatial, and temporal dynamics may be tested in the presence of known immune activators such as lipopolysaccharide (LPS), amyloid beta (AP42), and human immunodeficiency virus (HIV) glycoprotein (GP120) within appropriate experimental systems such as human microglia (HMC3) cells, that is established model for neuroimmune disease.

Conventional technologies for detecting oxidative stress, and inflammation in both research and clinical diagnosis often involve microscopy, immunoassays, or biochemical tests to manually measure specific markers (e.g., ROS) or morphological changes. While these methods have significantly contributed to advancing the understanding of inflammatory processes and are routinely used in diagnostic laboratories, they are frequently limited by 1) strong subjective interpretation; 2) time and labor intensity; 3) limitations that are inherent to the biochemical assay and its analysis. In clinical settings, these conventional approaches may also be constrained by variability in sample preparation, staining, and analysis, which can impact the reliability and reproducibility of diagnostic results. Traditional methods for analyzing cellular inflammation and oxidative stress in drug screening are often challenged by manual interpretation and difficulties in distinguishing subtle or heterogeneous responses. Moreover, the complexity and volume of data generated by high-content imaging can quickly surpass the capacity of human analysis, making the process of identifying nuanced patterns or correlations that may hold importance for understanding drug effects more difficult. Subtle changes in cellular morphology, spatial distribution of metabolic markers, or dynamic responses over time may go unnoticed or be interpreted inconsistently by human observers.

As can be seen, there is a need for improved approaches that can address the limitations of conventional analysis and provide more comprehensive, objective, and scalable assessments of compounds during drug discovery and inflammation research. In particular, there is a need for technologies that can determine the effectiveness of candidate compounds by objectively analyzing salient features of the cellular response during inflammation. Advanced imaging coupled with predictive platforms can accelerate high-throughput drug development and increase the reliability of preclinical screening.

### SUMMARY OF THE INVENTION

An embodiment of the present invention includes a method for predictive modeling of cellular samples. In the present invention, a cellular sample, treated with a compound, is exposed to at least one metabolic sensor(s), and the cellular sample is imaged to form one or more images. The one or more images are analyzed, using a predictive model, to determine an effectiveness of the compound on a metabolic state of the cellular sample.

In some embodiments, the at least one metabolic sensor(s) is one or more fluorescent probes for detecting oxidative stress of the cellular sample; visualizing a cytoskeleton organization of the cellular sample; or visualizing the location, morphology, or movement of mitochondria in the cellular sample.

In some embodiments, the one or more fluorescent probes is selected from the group consisting of: a cellular ROS (CellROX) probe; mitochondrial detection MitoYFP probe; mitochondrial membrane voltage sensor TMRE probe; mitochondrial ROS MitoROS probe; a cytoskeletal detector probe; and any combination thereof.

In some embodiments, the method further includes pre-processing, using one or more modules, the one or more images in preparation for analysis using the predictive model, wherein pre-processing includes one or more of: unpacking the one or more images into a flattened format; normalizing a value for each pixel in each of the one or more images; reducing noise from each of the one or more images; or reducing structural features of each of the one or more images.

In some embodiments, the metabolic state is one or more of: an immunological state of the cellular sample; an inflammatory state of the cellular sample; a bio-energetic state of the cellular sample; an oxidative stress state of the cellular sample; toxicity information of the cellular sample; or a proliferative information of the cellular sample.

An embodiment of the present invention includes a system for predictive modeling of cellular samples. The system includes an image capture device, at least one processor, and at least one memory storing instructions that when executed by the processor perform a method. The method captures one or more images of a cellular sample using the image capture device, when the cellular sample is exposed to at least one metabolic sensor. The one or more images are analyzed, using a predictive model, to determine an effectiveness of a compound on a metabolic state of the cellular sample.

In some embodiments, the predictive model of the system is trained by capturing, using the image capture device, one or more training images of a cellular sample, wherein the cellular sample is exposed to at least one metabolic sensor and at least one challenge condition. The training images are pre-processed in preparation for training the predictive model. Preprocessing of the training images can be performed by one or more modules such as an unpacking module configured to flatten the one or more images from a first format to a second format; a normalization module configured to normalize a value for each pixel in each of the one or more images; an augmentation module configured to generate one or more additional training images from the one or more training images; and a skeletonization module to provide morphological processing and skeletonization for each of the one or more images.

In some embodiments, at least one metabolic sensor is one or more fluorescent probes for: detecting oxidative stress the cellular sample; visualizing a cytoskeleton organization of the cellular sample; or visualizing the location, morphology, or movement of mitochondria in the cellular sample.

In some embodiments, the one or more fluorescent probes is selected from the group consisting of: a Reactive Oxygen Species probe; a MitoYFP probe; a TMRE probe; a MitoROS probe; and any combination thereof.

In some embodiments, the method of the system pre-processes the one or more images in preparation for analysis using the predictive model. The pre-processing can be performed by one or more modules such as an unpacking module configured to flatten the one or more images from a first format to a second format; a normalization module configured to normalize a value for each pixel in each of the one or more images; and a skeletonization module to provide morphological processing and skeletonization for each of the one or more images.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic diagram of a process and workflow for AI-driven cellular analysis and drug development using mitochondrial and oxidative combinations of redox sensors in combination with machine learning to create a fingerprint, according to aspects of the present invention;

FIG. 2 is a schematic diagram providing an overview of the cell imaging and predictive modeling of system, according to aspects of the present invention;

FIG. 3 is a flow diagram of a method for predictive modeling of cellular systems, according to aspects of the present invention;

FIG. 4A illustrates performance metrics of a first predictive model for predictive modeling of cellular systems, according to aspects of the present invention;

FIG. 4B illustrates performance metrics of a first predictive model for predictive modeling of cellular systems, according to aspects of the present invention;

FIG. 4C illustrates performance metrics of a first predictive model for predictive modeling of cellular systems, according to aspects of the present invention;

FIG. 5 illustrates performance metrics of a second predictive model for predictive modeling of cellular systems, according to aspects of the present invention;

FIG. 6 illustrates performance metrics of a third predictive model for predictive modeling of cellular systems, according to aspects of the present invention;

FIG. 7A illustrates representative images of MRX Sensors, according to aspects of the present invention;

FIG. 7B illustrates representative images of MRX Sensors, according to aspects of the present invention;

FIG. 7C illustrates representative images of MRX Sensors, according to aspects of the present invention; and

FIG. 7D illustrates representative images of MRX Sensors, according to aspects of the present invention.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Microglia are the primary immune cells of the central nervous system (CNS) and are key to overall maintenance of CNS health and responses to inflammation. Microglia function to perform maintenance of the CNS by constantly searching for plaques, damaged neurons and synapses, and/or infectious agents. Microglia have fine-grained sensitivity to pathological changes in the CNS due to the necessity of preventing fatal damage. Due to this sensitivity, microglial function is a hallmark of neural disease including neurodegenerative and neuroimmune conditions.

It is widely accepted that microglia transition between two primary functional states termed M1 and M2 that represent inflammatory and anti-inflammatory status, respectively. Microglial state transitions are accompanied by a metabolic switch to meet the changing energy demands of the cell. In fact, a defining feature of microglial activation is the close coupling between immunological function and cellular metabolism involving energy production and oxidative ROS properties. For example, pro-inflammatory, M1-like microglia shift toward glycolysis, fatty acid synthesis, and the pentose phosphate pathway that is often associated with greater ROS production, while anti-inflammatory, M2-like microglia rely on oxidative phosphorylation (OXPHOS) to support tissue repair and resolution of inflammation that is often associated with relatively lower ROS.

Mitochondria are central regulators of the metabolic switch of microglia and their network morphology (e.g., branching and elongation) is highly associated with the immune cell state. Generally, fission events produce fragmented mitochondria that promote glycolytic metabolism and pro-inflammatory signaling, whereas fused and elongated networks enhance OXPHOS and antioxidant capacity leading to lowered ROS. This network morphology is a key determinant of switch activation and reflects the metabolic and functional oxidative state of the microglia.

Conventional techniques for detecting inflammatory states, especially in immune cells such as microglia, typically rely on manual microscopy, immunoassays, or biochemical measurements of selected protein markers. These methods are limited by subjective interpretation, operator-dependent variability, and the need for skilled personnel, as well as the requirement to confirm results across multiple markers.

Furthermore, traditional approaches for assessing inflammation and oxidative stress during drug screening are constrained by manual data analysis and often lack the sensitivity to resolve subtle or heterogeneous cellular responses.

Broadly, an embodiment of the present invention provides an integrated platform for predictive simulation and modeling of cellular systems in the context of drug discovery and inflammatory oxidative state responses. The system includes assays of cellular samples exposed to a multi-sensor panel of mitochondrial functional and redox (MRX) imaging probes and at least one challenge condition. Additionally, each of the assays of cellular samples can be exposed to a compound. The system also includes an image capture device for capturing images and image processing modules for pre-processing the images for analysis by or training of predictive model(s). The predictive model(s) include Artificial Intelligence (AI) Model(s), or Machine Learning (ML) Model(s), for analyzing the pre-processed images to predictively classify the pre-processed images according to one or more criteria, such as efficacy of the compound, which can include both qualitative and quantitative values, and/or the type of challenge condition present. The predictive models are trained on images of cellular samples having one or more MRX imaging probes and the compound and/or at least one challenge condition resulting in an MRX compound-specific fingerprint.

Broadly, an embodiment of the present invention provides a method for predictive simulation and modeling of cellular systems in the context of drug discovery and inflammatory oxidative state responses through integrated MRX and AI image detection and analysis. The method creates assays of cellular samples exposed to at least one metabolic MRX imaging sensor as a probe and at least one challenge condition.

Additionally, each of the assays of cellular samples can be exposed to a compound. The method images, or receives images of, the assays, and pre-processes the images for analysis by or training of predictive model(s). The predictive model(s) analyze the pre-processed images to predictively classify the pre-processed images according to one or more criteria, such as efficacy of the compound, and/or the type of challenge condition present. The predictive model(s) are trained on images of cellular samples having the at least one metabolic sensor, the compound and/or the at least one challenge condition.

Broadly, in the context of the present invention cellular samples include samples of neural or immune cells, such as, but not limited to, neurons, microglia, macrophages, astrocytes, oligodendrocytes, T cells, B cells, dendritic cells, and natural killer (NK) cells of various source or origin, as well as primary cultures, immortalized cell lines, stem-cell-derived populations, or tissue-derived ex vivo preparations relevant to inflammatory, metabolic, or neuroimmune signaling.

Broadly, in the context of the present invention the compound is a molecular compound, such as, but not limited to, treatment compounds, compounds known or suspected to have anti-inflammatory properties including lipopolysaccharide, gangliosides, amyloids, viral glycoproteins, neurotoxic agents including taxanes, nicotinic agonists and antagonists (e.g., nicotine, acetylcholine, cytisine, varenicline), mitochondrial stressors (e.g., rotenone, antimycin A, CCCP, oligomycin), ROS modulators (e.g., hydrogen peroxide, menadione, tert-butyl hydroperoxide), phytocannabinoids (e.g., cannabidiol, cannabigerol, A9-tetrahydrocannabinol), synthetic cannabinoids, cytokines (e.g., TNF-α, IL-1β, IFN-γ), pattern-recognition receptor ligands (e.g., poly I:C, CpG DNA), inflammasome activators (e.g., nigericin, ATP), chemotherapeutic agents (e.g., paclitaxel, cisplatin), environmental toxicants (e.g., BMAA, heavy metals), and lipid-derived mediators (e.g., ceramides, prostaglandins). These compounds may elicit oxidative stress, mitochondrial remodeling, cytokine release, or other measurable inflammatory signatures in cellular systems relevant to the present invention.

Broadly, in the context of the present invention the at least one metabolic MRX imaging sensor is a probe, or marker, such as a fluorescent probe for detecting oxidative stress through ROS, visualizing the structural organization of the cell, and/or visualizing the location, morphology, and/or movement of the mitochondria network.

Broadly, in the context of the present invention the least one challenge condition is a compound, molecule, etc., having qualities that perturb the cellular samples through mitochondrial OXPHOS disruption or oxidative stress. More specifically, the at least one challenge condition can be a compound, such as a protein, lipid, or other compound, having known, or suspected, inflammatory qualities.

Broadly, in the context of the present invention the image capture device includes devices for capturing cellular images, such as cameras, microscopes, focal microscopes, etc.

Broadly, in the context of the present invention the image processing modules include modules for segmenting, flattening, normalizing, augmenting, and/or skeletonizing images.

Broadly, in the context of the present invention the predictive model(s) of the present invention are model(s) for predictive analysis and/or classification of image. More specifically, the predictive model(s) of the present invention can be neural networks, such as, but not limited to Feedforward Neural Networks, Convolutional Neural Networks (CNNs), Recurrent Neural Networks (RNNs), Transformer Networks, Generative Models, Autoencoders, Memory-Augmented Networks, Graph Neural Networks (GNNs), Attention-Based Networks, Siamese and Metric Learning Networks, Specialized Architectures, and/or Hybrid Architectures of one or more of the above.

Broadly, in the context of the present invention output(s) of the predictive model(s) are predictive classifications associated with the cellular sample under analysis. More specifically, predictive classifications include, but are not limited to, predictions related to the efficacy of the compound, one or more properties of the compound, predictions associated with the at least one challenge condition, and/or predictions associated with cellular sample responses. Broadly, the predictive model(s) of the present invention are trained, tested, and validated on images of cellular samples captured by the imaging device of the system. Broadly, in preparation for training, testing, and validation of the predictive model(s) cellular samples are transfected with a challenge condition, known to cause an inflammatory response, and further transfected with at least one or a combination of MRX imaging fluorescent probes. The at least one metabolic sensor is chosen based on a property of the cellular sample desired to be analyzed, predicted, or determined. Additionally, if the predictive model is for determining efficacy of a compound the cellular samples are segregated into treatment groups, and control groups, wherein the treatment groups were treated, either prior to transfection, or after transfection, with the compound.

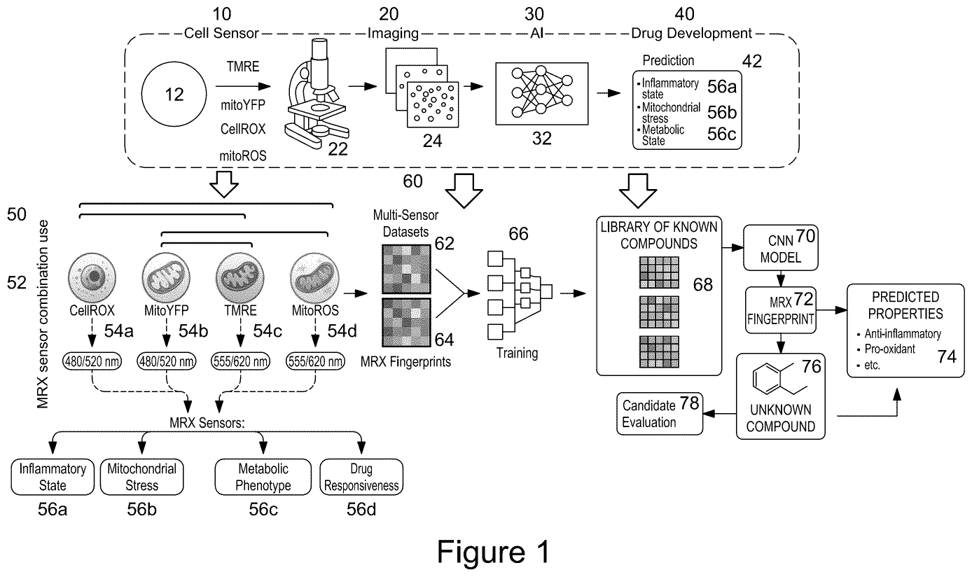

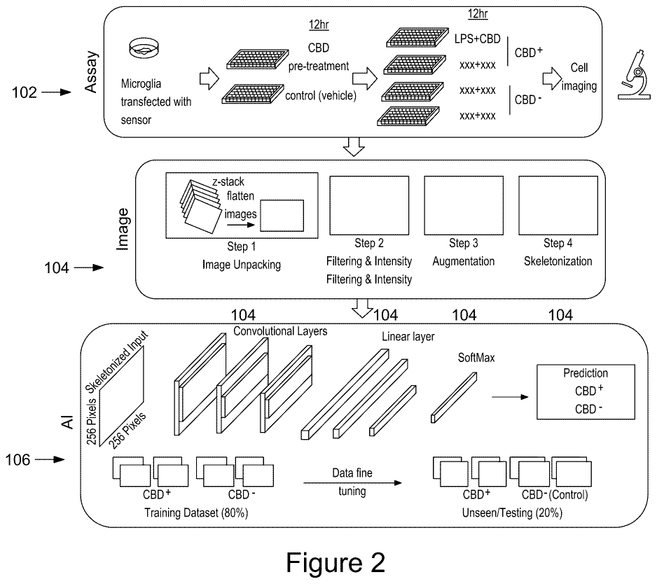

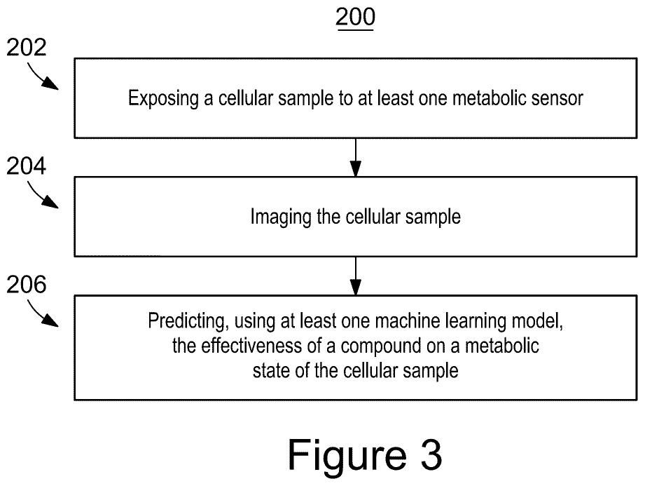

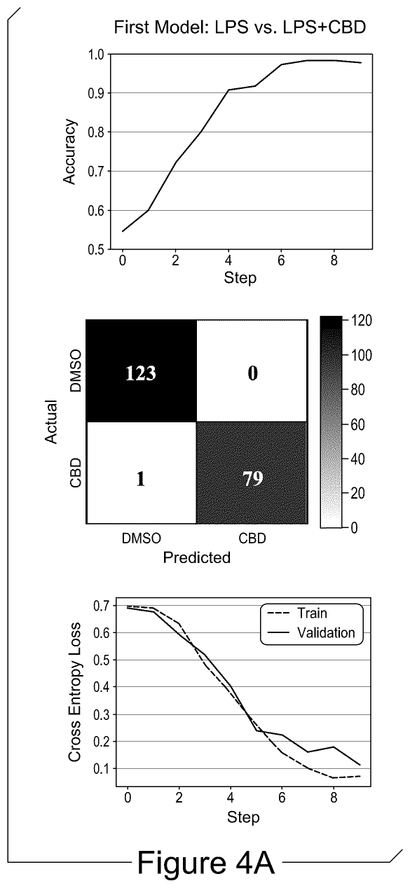

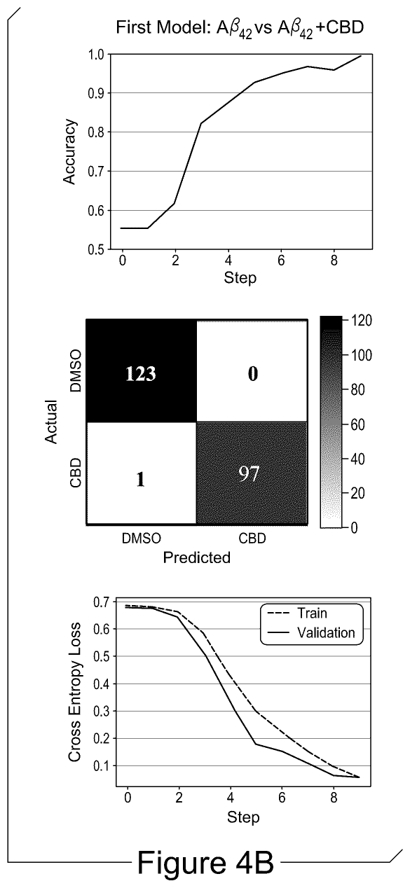

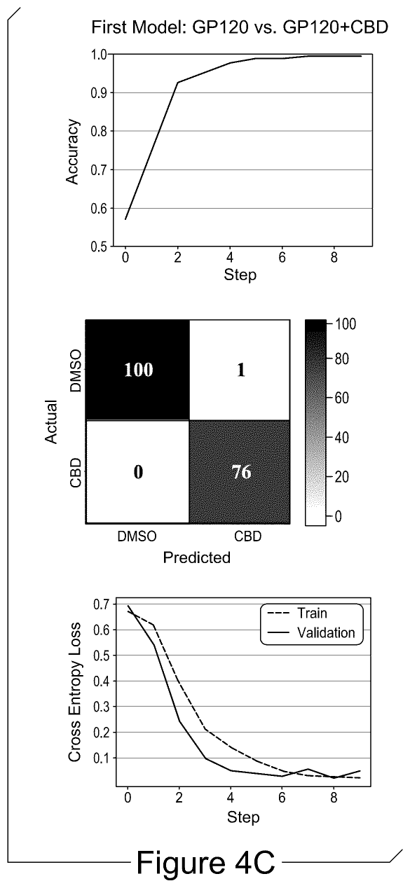

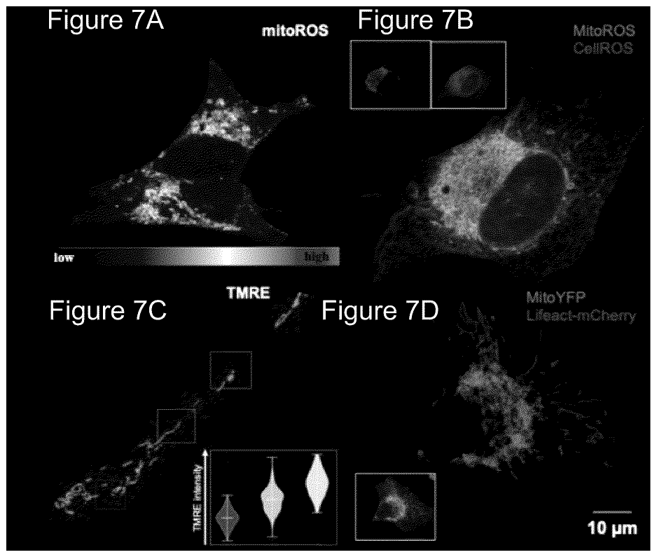

Referring now to the Figures, aspects of a system and method for predictive modeling of cellular systems are illustrated. Briefly, and described in more detail with reference to the accompanying Figures, FIG. 1 depicts the overall workflow for artificial intelligence-driven cellular analysis using mitochondrial functional and redox (MRX) sensors, FIG. 2 shows the system architecture with its components and modules, FIG. 3 illustrates the method flow diagram, and FIGS. 4-6 present model performance results including accuracy curves, confusion matrices, and loss curves for the predictive models. FIG. 7 provides examples of MRX sensor combinations within cells.

FIG. 1 depicts a four-stage process comprising cell sensing 10, imaging 20, artificial intelligence analysis 30, and drug development 40.

In the cell sensor stage 10, a cell 12 may be genetically modified to express one or more metabolic MRX imaging sensor 14, such as tetramethylrhodamine ethyl ester (TMRE), MitoYFP, CellROX, and MitoROS.

In the imaging stage 20, an image capture device, such as a microscope 22, captures fluorescent signals from the metabolic sensors in a plurality of fluorescent microscopy images 24 of sensor-modified cells as illustrated in FIG. 7, captured after the cells have been subjected to stress conditions. Some images within plurality of images 24 represent cells treated with test compounds, while other images may represent control cells.

Neural network 32 in the artificial intelligence stage 30 analyzes the plurality of images 24 to identify patterns associated with different cellular states.

Prediction output 42 in the drug development stage 40 contains three cellular state classifications: inflammatory state 56a, mitochondrial stress 56b, and metabolic state 56c. This prediction output connects to and provides input for MRX sensor workflow 50.

The MRX sensor workflow 50 begins with MRX sensor combinatorial use 52. Sensor-specific cell images—CellROX 54a, MitoYFP 54b, TMRE 54c, and MitoROS 54d—are typically but not exclusively associated with corresponding excitation/emission wavelengths: 480/520 nm for CellROX 54a, 480/520 nm for MitoYFP 54b, 555/620 nm for TMRE 54c, and 555/620 nm for MitoROS 54d.

MRX sensor outputs 56 comprise four measurement categories: inflammatory state 56a, mitochondrial stress 56b, metabolic phenotype 56c, and drug responsiveness 56d.

Data processing module 60 comprises multi-sensor datasets 62 and MRX fingerprints 64. Training module 66 represents the training process for the neural network.

Compound library 68 contains known compounds with characterized properties. CNN model 70 is a convolutional neural network model trained on imaging data from the known compounds to predict cellular properties of unknown compounds.

MRX fingerprint data 72 is used to predict properties, for example anti-inflammatory or pro-oxidant properties, shown in predicted properties output 74. For unknown compounds 76, the MRX fingerprint 72 is used to determine predicted properties 74 to inform its evaluation as a drug candidate in candidate evaluation module 78.

FIG. 2 illustrates a schematic diagram of a System 100 for predictive modeling of cellular systems, according to aspects of the present invention. Briefly, and described in more detail below, System 100 has a plurality of components for use in predictive modeling of cellular systems, such as Assay(s) 102, Image(s) 104, and Predictive Model(s) 106.

The Assay(s) 102 result in cellular samples that are perturbed by challenge agent(s), such as agent(s) known to cause inflammatory response(s) therein, and/or are either treated with compound(s) having, or thought to have, anti-inflammatory capabilities, or with a control molecular compound.

In embodiments, the challenge agent is one or more of: Human Immunodeficiency Virus (HIV) protein GP120, Amyloid Beta protein Aβ₂₄, or Lipopolysaccharide (LPS), but is not so limited. In embodiments, the compound is a non-psychoactive Cannabinoid, such as CBD, but is not so limited.

The cellular samples are exposed, or transfected, with at least one or a mixture of MRX imaging probes for sensing metabolic and oxidative conditions of the cellular samples. In embodiments, the MRX imaging probes are one or more fluorescent probes for detecting oxidative stress, visualizing the cytoskeleton organization of a cell, and/or visualizing the location, morphology, and/or movement of mitochondria. In exemplary embodiments, the metabolic MRX sensor is one or more of: Lifeact-mCherry, CellROX, MitoROS, TMRE, or MitoYFP.

In embodiments, Lifeact-mCherry is a genetically encoded fluorescent sensor that binds to filamentous actin (F-actin) with high specificity, enabling visualization of cytoskeletal organization and dynamic actin remodeling in living cells. Lifeact-mCherry serves as a reporter of morphological and structural changes associated with cellular activation, motility, and inflammatory signaling. During immune activation or stress responses, alterations in actin polymerization and cytoskeletal architecture are hallmarks of cellular state changes; therefore, Lifeact-mCherry provides a real-time indicator of cytoskeletal dynamics that correlate with inflammatory or metabolic perturbations.

In embodiments, CellROX is a fluorogenic oxidative stress probe that exhibits increased fluorescence upon oxidation by reactive oxygen species (ROS), including superoxide and hydroxyl radicals. When applied to living cells, CellROX accumulates in specific intracellular compartments and becomes brightly fluorescent in the presence of elevated oxidative stress. CellROX therefore enables quantitative detection of ROS-associated inflammatory states, mitochondrial dysfunction, or xenobiotic-induced oxidative injury, providing a sensitive readout of redox imbalance in the cellular samples.

In embodiments, MitoYFP is a mitochondrially targeted fluorescent protein that localizes to the mitochondrial matrix or inner membrane space, enabling visualization of mitochondrial morphology, distribution, and network dynamics. MitoYFP reports on mitochondrial health by revealing fragmentation, elongation, swelling, or redistribution events associated with metabolic stress, calcium dysregulation, or inflammatory activation. Because mitochondrial architecture is tightly linked to oxidative phosphorylation efficiency and ROS generation, MitoYFP provides a high-content imaging marker for mitochondrial state changes that occur during cellular inflammation or exposure to test compounds.

In embodiments, MitoROS is a mitochondria-targeted fluorescent probe that selectively detects ROS generated within the mitochondrial compartment. Upon oxidation, MitoROS exhibits increased fluorescence intensity, providing a sensitive and compartment-specific measure of mitochondrial redox status. Because mitochondrial ROS production is a key driver of inflammatory signaling, metabolic dysfunction, and cell stress responses, MitoROS enables precise quantification of mitochondrial oxidative burden in living cells. The probe allows high-content imaging of mitochondrial ROS dynamics, including responses to inflammatory stimuli, xenobiotic compounds, or metabolic stressors, thereby serving as an indicator of mitochondrial integrity and function within the cellular samples.

In embodiments, TMRE (tetramethylrhodamine ethyl ester) is a mitochondria-permeant fluorescent probe that selectively accumulates in active mitochondria in proportion to their membrane potential. Upon uptake into polarized mitochondria, TMRE exhibits robust fluorescence, providing a sensitive and real-time measure of mitochondrial bioenergetic status. Because mitochondrial membrane potential is a central indicator of metabolic activity, apoptotic resistance, and organelle function, TMRE enables precise quantification of mitochondrial polarization in living cells. The probe supports high-content imaging of mitochondrial functional dynamics, including depolarization events, drug-induced mitochondrial stress, or shifts in cellular metabolic state, thereby serving as a core indicator of mitochondrial health and overall cellular metabolic integrity within the samples.

The perturbed, treated, and transfected cellular samples are captured, or visualized, as image(s) 104 using an image capture device 102a, such as a camera, microscope, or other image capture device. In embodiments, the image(s) 104 are captured utilizing a confocal microscope, such as a Zeiss LSM 800, using confocal microscopy techniques, and filtering based on the at least one, or a combination of MRX sensor probes utilized. In embodiments, a 480/520 nanometer (nm) filter is utilized for CellROX sensor(s) or MitoYFP sensor(s), and a 555/620 nm filter is utilized for Lifeact-mCherry, TMRE, or MitoROS sensor(s). In embodiments, the image(s) 104 are captured as z-stack images wherein images of the cellular samples are taken in optical sections varied along the z-axis, thereby forming a three-dimensional image of the cellular samples using a plurality of two-dimensional image sections.

The Image(s) 104, once captured by the image capturing device 102a, are pre-processed by system 100 in preparation for training, testing, and validation of Predictive Model(s) 106. System 100 includes one or more modules for pre-processing image(s) 104. The one or more modules can include: an image unpacking module 104a, a normalization module 104b, an augmentation module 104c, and/or a skeletonization module 104d. It is understood that System 100 can include all, or a portion of the one or more modules, and the one or more modules can be rearranged in order of processing.

The unpacking module 104a is configured to unpack image(s) 104 from a three-dimensional representation to a plurality of two-dimensional representations. In embodiments, each image(s) 104 as a z-stack includes a plurality of two-dimensional images combined to form one three-dimensional image. In embodiments, the unpacking module 104a flattens each image(s) 104 into separate files for each of its constituent two-dimensional images for further processing.

The normalization module 104b is configured to convert image(s) 104 to a standard representation to account for variability. In embodiments, normalization module 104b normalizes each pixel value of each image(s) 104 to a value between 0-255, according to the following equation:

$${pixel\_value}_{new} = \frac{( {pixel\_value}_{old} - {pixel\_value}_{minimum} )}{( {pixel\_value}_{maximum} - {pixel\_value}_{minimum} )} * 255.$$

In addition, normalization module 104b converts each image to greyscale, after normalization, and/or reduces dimensions of each image(s) 104. In embodiments, normalization module 104b reduces each image(s) 104 dimensions to a standard size, such as 256 pixels×256 pixels. Additionally, normalization module 104b performs enhancement of the contrast of image(s) 104 using one or more functions, such as histogram equalization, to maximize an intensity range of pixel values for image(s) 104.

The augmentation module 104c is configured to expand the corpus of image(s) 104 by generating synthetic image(s) using image(s) 104. In embodiments, augmentation module 104c rotates each image(s) 104 one or more times to form one or more synthetic images. In exemplary embodiments, augmentation module 104c rotates each image(s) 104 at 0°, 90°, 180°, and 270°, creating at least four synthetic images from each image(s) 104, thereby increasing the corpus useful for training, testing, and validation of Predictive Model(s) 106.

The Skeletonization module 104d is configured to provide morphological processing and skeletonization of image(s) 104 thereby reducing/removing noise and/or to simplify structural details of image(s) 104 while preserving useful information, such as cellular topology. In embodiments, morphological processing includes noise reduction by erosion and dilation of image(s) 104. In an exemplary embodiment, morphological processing is performed using one or more library functions, such as morphology.opening function available from the scikit-learn library. In embodiments, skeletonization of image(s) 104 reduces any structure in each image(s) 104 to single pixel width centerlines while keeping the topology of the image(s) 104 intact. In an exemplary embodiment, skeletonization is performed using one or more library functions, such as morphology. skeletonize function available from the scikit-learn library.

The Predictive Model(s) 106 are trained, tested, and validated using image(s) 104 to predict cellular condition(s) of images provided thereto. In embodiments, the Predictive Model(s) 106 include one or more neural networks, such as convolutional neural networks, configured to predict cellular condition(s) of images. Predictive Model(s) 106 are trained, tested, and validated on using image(s) 104 associated with the type of cellular condition sought to be predicted.

In embodiments, Predictive Model(s) 106 are trained, tested and validated on Microglial cells, such as human Microglial cells, being the cellular samples. The Microglial cells are transfected using the challenge condition, known to cause inflammatory response, such as Human Immunodeficiency Virus (HIV) protein GP120, Amyloid Beta protein Aβ₂₄, or Lipopolysaccharide (LPS). The transfected cells are segregated into treatment groups, and control groups, wherein the treatment groups were treated, either prior to transfection, or after transfection, with a compound thought, or demonstrated, to have anti-inflammatory capabilities, such as Cannabidiol (CBD).

The transfected cells are further transfected with, or exposed to, at least one or a combination of MRX sensors. In embodiments, the MRX sensors comprise one or more fluorescent or luminescent probes capable of reporting oxidative stress, redox state, cytoskeletal organization, mitochondrial location, mitochondrial membrane potential, mitochondrial morphology, or mitochondrial dynamics. In exemplary embodiments, the metabolic sensor includes, but is not limited to, probes such as Lifeact-mCherry, CellROX, MitoROS, MitoYFP, or functionally equivalent sensors.

In further embodiments, two or more MRX sensors are used in combination to generate a multiplexed readout of cellular state. For example, the simultaneous use of MitoROS and CellROX enables detection of both compartment-specific mitochondrial ROS and global cellular oxidative stress, producing a unique, multidimensional fingerprint of inflammatory activation, mitochondrial dysfunction, and redox imbalance. Such combinatorial sensor profiling enhances sensitivity and specificity in identifying metabolic signatures associated with inflammation or exposure to test compounds

In embodiments, when two or more MRX sensors are used concurrently, the system employs spectral unmixing to resolve overlapping emission spectra of the probes. Spectral unmixing enables accurate separation, quantification, and spatial mapping of individual sensor signals within a single imaging frame. This process permits simultaneous detection of global oxidative stress, compartment-specific mitochondrial ROS, cytoskeletal remodeling, and mitochondrial structural changes without fluorescent interference, thereby improving the fidelity and resolution of the multiplexed metabolic measurements.

In embodiments, the combined signals from two or more MRX sensors including, but not limited to, CellROX, MitoROS, MitoYFP, or equivalent redox- and mitochondria-targeted probes—are computationally integrated to generate a reactive oxygen species (ROS) fingerprint. The ROS fingerprint comprises a multidimensional profile of cellular oxidative states derived from spatial distribution, fluorescence intensity, temporal dynamics, and compartment-specific ROS production. In further embodiments, the ROS fingerprint is used to classify inflammatory states, determine mitochondrial dysfunction, or identify responses to candidate compounds, optionally using machine-learning-based or statistical pattern-recognition algorithms.

Images of both the treated and control transfected cells are captured, pre-processed, and input into Predictive Model(s) 106 for testing, training, and validation of the model. Predictive Model(s) 106, once validated, are configured to perform predictive classification to determine the presence or absence of a treatment condition, i.e. the compound, detect the presence of one or more of the challenge conditions, and/or [add other predictive capabilities here], in Microglial cells.

In a first exemplary embodiment, a first predictive model of Predictive Model(s) 106 is configured to predict a treatment condition, such as the presence or absence of a treatment condition, i.e. a compound, in images being transfected with at least one metabolic sensor, as described above.

In embodiments, the first set of predictive models classify images as being treated with CBD(+), or a control CBD(−). The first set of models utilizes two convolutional layers and three linear layers to classify images having the CellROX metabolic sensor as being treated with CBD (positive) or control (negative). The first convolutional layer processes a 256×256 pixel image using eight filter maps with a kernel size equal to 16. A 5×5 max pooling layer is applied to a 0.20 dropout layer to prevent overfitting. The input to the second convolutional layer is 8×48×48 and uses 16 filter maps with a kernel size of 5. Successive dense linear layers are used to reduce the input image from 1×1024 down to 1×2. The final 2-class categorization provides a prediction of the presence or absence of CBD (+/−) using a SoftMax function on one or more images of cellular samples provided thereto.

In a second exemplary embodiment, a second predictive model of Predictive Model(s) 106, differs from the above models' use 2-class categorization to predict conditions that distinguish the presence or absence of a treatment condition, i.e. the compound, across the three perturbed challenge conditions (LPS, GP120, and Aβ₄₂), and distinguish between the three challenge conditions.

In embodiments, the second predictive model utilizes two convolutional layers and three linear layers to classify images as being perturbed by LPS, GP120, or Aβ₄₂. A The first convolutional layer processes a 256×256 pixel image using eight filter maps with a kernel size equal to 16. In this case, a 5×5 max pooling layer was applied to a 0.20 dropout layer to prevent overfitting. The input to the second convolutional layer was 8×48×48 using 16 filter maps with a kernel size equal to 5. Successive dense linear layers are used to reduce the input from 1×1024 down to 1×3 and the final network ternary prediction, i.e. LPS, GP120, or Aβ₄₂, is computed using Softmax function on one or more images of cellular samples provided thereto.

In a third exemplary embodiment, a third predictive model of the Predictive Model(s) 106 is configured to predict a treatment condition, such as the presence or absence of a treatment condition, i.e. the compound, in images being transfected with a mitochondrial (MitoYFP) MRX sensor, as described above.

In embodiments, the third set of predictive models classify images as being treated with CBD(+), or a control CBD(−). The third set of models utilizes two convolutional layers and three linear layers to classify images having the MitoYFP metabolic sensor as being treated with CBD (positive) or control (negative). The models use three convolutional layers and three linear layers to classify MitoYFP images as being treated with CBD (CBD+) or control (CBD−). The first convolutional layer processes a 256×256 image using sixteen filter maps with a kernel size equal to 7. The next convolutional layers increase the number of filters to 32 and then to 64, each followed by activation and pooling operations to shrink the spatial dimensions. This flattened output is passed through successive dense linear layers to reduce the input from 1×1024 down to 1×128 and then to 1×2. The final binary prediction of the CBD+ or CBD− condition is computed using Softmax function on one or more images of cellular samples provided thereto.

The Predictive Model(s) 106 are trained, validated, and tested using image(s) 104 split into training, validation, and testing groups. An optimization function utilizing a loss function is utilized to optimize each of Predictive Model(s) 106. A hyperparameter controls the rate at which the loss function is minimized by the optimization function. In embodiments, image(s) 104 are split into data sets by percentage, such as 72/8/20 for training, validation, and testing. In embodiments, the optimization function is an ADAM optimizer, which utilizes a loss function, such as cross-entropy loss. Additionally, the hyperparameter, learning rate, for the ADAM optimizer is set to 0.0001 to control the size of updates made to the Predictive Model(s) 106 weights, during training.

In the first exemplary embodiment, the first predictive model is trained using image(s) 104 having cellular samples perturbed with LPS, GP120, or Aβ₄₂ and having a treatment condition of CBD (+) or control CBD(−). In this exemplary embodiment, the first predictive model includes three variations. Model performance results for each group are presented in FIG. 4 as accuracy curves, confusion matrices, and loss curves. Model accuracy is used to measure model performance based on its ability to correctly classify the image as CBD(+) or control CBD(−). Results show all the first model achieved >90% accuracy by the fifth epoch and >99% accuracy by the tenth epoch. Confusion matrices illustrate the occurrence of false positive and false negative outcomes. These matrices show high predictive model performance. Lastly, the cross-entropy loss method, which measures loss on a 0-1 scale with 0 reflecting perfect model performance. As indicated in FIG. 4, convergence of the training and validation loss curves suggests little to no overfitting within the three models.

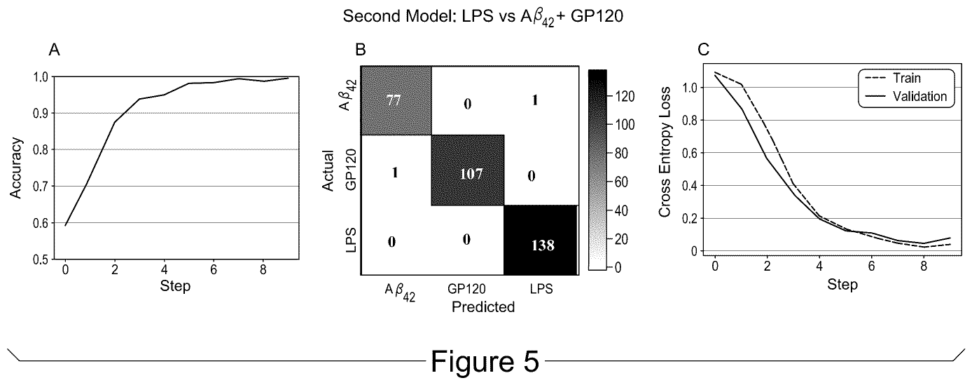

In the second exemplary embodiment, the second predictive model is trained using image(s) 104 having cellular samples perturbed with LPS, GP120, or Aβ₄₂. Model performance results are presented in FIG. 5 as accuracy curves, confusion matrices, and loss curves. Model accuracy is used to measure model performance based on its ability to correctly classify the image as CBD (+) or control CBD(−). Results show all the first model achieved >90% accuracy by the fifth epoch and >99% accuracy by the tenth epoch. Confusion matrices were generated to examine the occurrence of false positive and false negative outcomes. These matrices show high predictive model performance. Lastly, the cross-entropy loss method, which measures loss on a 0-1 scale with 0 reflecting perfect model performance. As indicated in FIG. 5, convergence of the training and validation loss curves suggests little to no overfitting within the model.

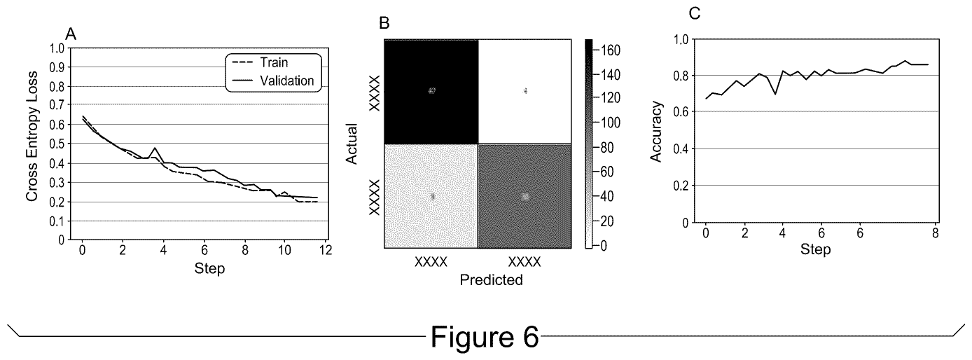

In the third exemplary embodiment, the third predictive model is trained using image(s) 104 having cellular samples cells that were treated with LPS and Aβ₄₂ comparing treatment with CBD+ or CBD− conditions. Model performance results are presented in FIG. 6 as accuracy curves, confusion matrices, and loss curves. Model accuracy is used to measure model performance based on its ability to correctly classify the image as CBD (+) or control CBD(−). Results show the model achieved >82% accuracy by the 25^{th} epoch. Confusion matrices were generated to examine the occurrence of false positive and false negative outcomes. These matrices show high predictive model performance. Lastly, the cross-entropy loss method, which measures loss on a 0-1 scale with 0 reflecting perfect model performance. As indicated in FIG. 6, convergence of the training and validation loss curves suggests little to no overfitting within the model.

FIG. 3 illustrates a flow diagram of method 200 for predictive modeling of cellular systems, according to aspects of the present invention. Briefly, and described in more detail below, method 200 exposes a cellular sample, such as a Microglia cell, to at least one or combinations of MRX sensors the result of the exposure is imaged using an image capturing device, and provided to at least one predictive model to predict an effectiveness of a compound, such as a treatment compound, on a metabolic state of the sample, and to further predict one or more states of the sample, such as: (i) a level of oxidative stress; (ii) mitochondrial dysfunction or altered mitochondrial dynamics; (iii) inflammatory activation state; (iv) cytoskeletal remodeling; (v) redox imbalance across cellular compartments; (vi) cellular toxicity or viability outcomes; (vii) dose-response relationships; (viii) mechanism-of-action pathways. Method 200, and functionality associated therewith, can be performed using System 100, and/or one or more of the modules therein, but is not so limited.

At step 202, a cellular sample is exposed to at least one MRX sensors the. The cellular sample is perturbed by a challenge condition, such as an inflammatory condition caused by challenge from a bacterial protein, etc. In embodiments the challenge condition is one or more of Human Immunodeficiency Virus (HIV) protein GP120, Amyloid Beta protein Aβ₂₄, or Lipopolysaccharide (LPS), but is not so limited. Additionally, the cellular sample is treated using a compound thought to affect the challenge condition of the cellular sample. In embodiments, the compound is a treatment compound thought to affect, or remediate, the challenge condition in the cellular sample. In embodiments, the compound is a non-psychoactive Phyto cannabinoid, such as cannabidiol (CBD), but is not so limited as one or more combinations of compounds, formulations of compounds, etc., are equally contemplated. In embodiments, the cellular sample is a sample of CNS cells, such as Microglia cells, but is not so limited. In embodiments, the at least one metabolic sensor is one or more fluorescent probes for detecting oxidative stress, visualizing the cytoskeleton organization of a cell, and/or visualize the location, morphology, and/or movement of mitochondria. In exemplary embodiments, the MRX sensor is one or more of: Lifeact-mCherry, CellROX green, MitoROS, TMRE, or MitoYFP.

At step 204, imaging of the cellular sample is performed by an image capture device, such as a camera, microscope, or other image capture device. In embodiments, imaging of the cellular sample utilizes a confocal microscope, such as a Zeiss LSM 800, using confocal microscopy techniques, and filtering based on the at least one MRX sensor utilized. For example, a 480/520 nanometer (nm) filter is utilized for CellROX sensor or MitoYFP sensor(s), and a 555/620 nm filter is utilized for Lifeact-mCherry or MitoROS sensor. In embodiments, imaging of the cellular sample is captured as z-stack images wherein images of the cellular sample are taken in optical sections varied along the z-axis, thereby forming a three-dimensional image of the cellular sample using a plurality of two-dimensional image sections.

Once imaging of the cellular sample has been performed, optional pre-processing of the resulting image can be performed. In embodiments, optional pre-processing includes one or more of image unpacking, image normalization, and/or image skeletonization. In exemplary embodiments, optional pre-processing is performed by System 100, specifically modules 104a-b, and 104d, as described above.

At step 206, the imaged cellular samples are analyzed by a predictive model to predict the effectiveness of the treatment compound on a metabolic state of the cellular sample to which it was applied. In embodiments, the predictive model comprises one or more computational, statistical, or machine-learning algorithms configured to extract quantitative features from the MRX sensor-derived images, including fluorescence intensity, spatial distribution, temporal dynamics, morphological changes, mitochondrial architecture, and patterns of redox activity. The predictive model predicts an effectiveness of the treatment compound, classified through an MRX fingerprint, on one or more of: an immunological state of the cellular sample, an inflammatory activation state of the cellular sample, an oxidative or bio-energetic state of the cell, and/or toxicity information, including apoptotic or necrotic signatures. In additional embodiments, the predictive model evaluates mitochondrial dysfunction, cytoskeletal remodeling, redox imbalance across cellular compartments, dose-response relationships, compound mechanism-of-action, or other phenotypic indicators of compound efficacy or safety.

In embodiments, the predictive model is one or more deep neural networks, such as one or more convolutional neural networks, trained, tested, and validated on cellular imaging data. Specifically, the predictive model is one or more of Predictive Model(s) 106, and is trained, tested, validated, and performs as described with respect thereto. In exemplary embodiments, the predictive model predicts the effectiveness of the treatment compound by analyzing the one or more images for signals generated by the at least one metabolic sensor, and further predicts one or more of: (i) oxidative stress levels; (ii) mitochondrial dysfunction or alterations in mitochondrial network topology; (iii) cytoskeletal remodeling; (iv) inflammatory activation state; (v) compound-induced toxicity; (vi) dose-response characteristics; (vii) mechanism-of-action profiles; (viii) phenotypic subclassification of cellular states; (ix) temporal or trajectory predictions of metabolic state changes; and (x) similarity scoring or fingerprint matching to known metabolic or inflammatory profiles.

In embodiments, the method further comprises computing, from the one or more images, a multidimensional metabolic or reactive oxygen species (ROS) fingerprint that characterizes the metabolic state of the cellular sample. The metabolic or ROS related MRX fingerprint comprises quantitative features derived from sensor signals, including fluorescence intensity, spatial distribution, compartment-specific redox activity, mitochondrial morphology, or temporal dynamics. In further embodiments, the metabolic or ROS related MRX fingerprint is compared to a plurality of reference MRX fingerprints stored in a database to identify similarities, differences, or deviations from known metabolic, inflammatory, or bio-energetic states. The method optionally includes storing the computed MRX fingerprint in the database for subsequent retrieval or matching, and identifying novel or hybrid metabolic states based on fingerprint patterns, thereby supporting compound screening, mechanism-of-action determination, or cellular pathway mapping

Embodiments of the invention and all of the functional operations described in this specification can be implemented in digital electronic circuitry, or in computer software, firmware, or hardware, including the structures disclosed in this specification and their structural equivalents, or in combinations of one or more of them. Embodiments of the invention can be implemented as one or more computer program products, e.g., one or more modules of computer program instructions encoded on a computer readable medium for execution by, or to control the operation of, data processing apparatus. The computer readable medium can be a machine-readable storage device, a non-transitory machine-readable storage device, a machine-readable storage substrate, a memory device, a composition of matter effecting a machine-readable propagated signal, or a combination of one or more of them. The term “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them. A propagated signal is an artificially generated signal, e.g., a machine-generated electrical, optical, or electromagnetic signal that is generated to encode information for transmission to suitable receiver apparatus.

A computer program (also known as a program, software, software application, script, or code) can be written in any form of programming language, including compiled or interpreted languages, and it can be deployed in any form, including as a standalone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of computer. Generally, a processor will receive instructions and data from a read only memory or a random access memory or both. The essential elements of a computer are a processor for performing instructions and one or more memory devices for storing instructions and data. Generally, a computer will also include or be operatively coupled to receive data from or transfer data to, or both, one or more mass storage devices for storing data, e.g., magnetic, magneto optical disks, or optical disks. However, a computer need not have such devices. Generally, a computer will also include a communications device. The communication device can include hardware and/or software for generating and communicating signals over a direct and/or indirect network communication link. As used herein, a direct link can include a link between two devices where information is communicated from one device to the other without passing through an intermediary. For example, the direct link can include a Bluetooth™ connection, a Zigbee connection, a Wifi Direct™ connection, a near-field communications (“NFC”) connection, an infrared connection, a wired universal serial bus (“USB”) connection, an ethernet cable connection, a fiber-optic connection, a firewire connection, a microwire connection, and so forth. In another example, the direct link can include a cable on a bus network. An indirect link can include a link between two or more devices where data can pass through an intermediary, such as a router, before being received by an intended recipient of the data. For example, the indirect link can include a WiFi connection where data is passed through a WiFi router, a cellular network connection where data is passed through a cellular network router, a wired network connection where devices are interconnected through hubs and/or routers, and so forth. The cellular network connection can be implemented according to one or more cellular network standards, including the global system for mobile communications (“GSM”) standard, a code division multiple access (“CDMA”) standard such as the universal mobile telecommunications standard, an orthogonal frequency division multiple access (“OFDMA”) standard such as the long term evolution (“LTE”) standard, and so forth.

Moreover, a computer can be embedded in another device, e.g., a tablet computer, a mobile telephone, a personal digital assistant (PDA), a mobile audio player, a Global Positioning System (GPS) receiver, to name just a few. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, embodiments of the invention can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

Embodiments of the invention can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the invention, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other.

While this specification contains many specifics, these should not be construed as limitations on the scope of the invention or of what may be claimed, but rather as descriptions of features specific to particular embodiments of the invention. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

It should be understood, of course, that the foregoing relates to exemplary embodiments of the invention and that modifications may be made without departing from the spirit and scope of the invention as set forth in the following claims.

What is claimed is:

1. A method comprising:
exposing a cellular sample, treated with a compound, to at least one metabolic sensor(s);
imaging the cellular sample to form one or more images; and
analyzing the one or more images, using a predictive model, to determine an effectiveness of the compound on a metabolic state of the cellular sample.

2. The method of claim 1, wherein the at least one metabolic sensor(s) is one or more fluorescent probes for:
detecting oxidative stress of the cellular sample;
visualizing a cytoskeleton organization of the cellular sample; or
visualizing the location, morphology, or movement of mitochondria in the cellular sample.

3. The method of claim 2, wherein the one or more fluorescent probes is selected from the group consisting of:
a Reactive Oxygen Species probe;
a MitoYFP probe;
a TMRE probe;
a MitoROS probe;
a cytoskeletal probe; and
any combination thereof.

4. The method of claim 1, further comprising:
pre-processing, using one or more modules, the one or more images in preparation for analysis using the predictive model, wherein pre-processing includes one or more of:
unpacking the one or more images into a flattened format;
normalizing a value for each pixel in each of the one or more images;
reducing noise from each of the one or more images; or
reducing structural features of each of the one or more images.

5. The method of claim 1, wherein the metabolic state is one or more of:
an immunological state of the cellular sample;
an inflammatory state of the cellular sample;
a bio-energetic state of the cellular sample;
an oxidative stress state of the cellular sample;
toxicity information of the cellular sample; or
a proliferative information of the cellular sample.

6. A system for predictive modelling of cellular systems, comprising:
an image capture device;
at least one processor; and
at least one memory storing instructions that when executed by the processor perform a method, the method comprising:
capturing, using the image capture device, one or more images of a cellular sample, wherein the cellular sample is exposed to at least one metabolic sensor(s);
analyzing the one or more images, using a predictive model, to determine an effectiveness of a compound on a metabolic state of the cellular sample.

7. The system of claim 6, wherein training the predictive model further comprises:
capturing, using the image capture device, one or more training images of a cellular sample, wherein the cellular sample is exposed to at least one metabolic sensor and at least one challenge condition;
pre-processing the one or more training images in preparation for training the predictive model, wherein pre-processing includes one or more of:
an unpacking module configured to flatten the one or more images from a first format to a second format;
a normalization module configured to normalize a value for each pixel in each of the one or more images;
an augmentation module configured to generate one or more additional training images from the one or more training images; or
a skeletonization module to provide morphological processing and skeletonization for each of the one or more images.

8. The system of claim 6, wherein the at least one metabolic sensor(s) is one or more fluorescent probes for:
detecting oxidative stress the cellular sample;
visualizing a cytoskeleton organization of the cellular sample; or
visualizing the location, morphology, or movement of mitochondria in the cellular sample.

9. The system of claim 8, wherein the one or more fluorescent probes is selected from the group consisting of:
a Reactive Oxygen Species probe;
a MitoYFP probe;
a TMRE probe;
a MitoROS probe; and
any combination thereof.

10. The system of claim 6, further comprising:
pre-processing the one or more images in preparation for analysis using the predictive model, wherein pre-processing includes one or more of:
an unpacking module configured to flatten the one or more images from a first format to a second format;
a normalization module configured to normalize a value for each pixel in each of the one or more images; and
a skeletonization module to provide morphological processing and skeletonization for each of the one or more images.
